(19) United States
(12) Patent Application Publication
(10) Pub. No.: US 2012/0165386 A1
(43) Pub. Date: Jun. 28, 2012

(54) STABLE ORAL PHARMACEUTIAL COMPOSITION OF ATORVASTATIN

(75) Inventors: Ravindra AGARWAL, Udaipur (IN); Parikshit Rameshrao KULKARNI, Solapur (IN); Ravi KOCHHAR, Gurgaon (IN)

(73) Assignee: RANBAXY LABORATORIES LIMITED, New Delhi (IN)

(21) Appl. No.: 13/334,452

(22) Filed: Dec. 22, 2011

(30) Foreign Application Priority Data
Dec. 27, 2010 (IN) 3113/DEL/2010

(51) Int. Cl. A61K 31/40

(52) U.S. Cl. 514/423

(57) ABSTRACT

The present invention relates to a stable oral pharmaceutical composition comprising atorvastatin or a pharmaceutically acceptable salt thereof and an alkalizing agent, wherein the buffering capacity of the composition is between 2 to 5.

### FIELD OF THE INVENTION

The present invention relates to a stable oral pharmaceutical composition comprising atorvastatin or a pharmaceutically acceptable salt thereof and an alkalizing agent, wherein the buffering capacity of the composition is between 2 to 5.

### BACKGROUND OF THE INVENTION

Atorvastatin belongs to a well-known group of drugs, statins, which are useful for the treatment of hypercholesterolemia or hyperlipidemia. Statins block the hydroxyl-methylglutaryl-coenzyme (HMG-CoA) reductase, thereby specifically inhibiting cholesterol synthesis in the liver. Although cholesterol is a vital component of all cells, it also contributes to plaque formation in arteries, which increases the risk of high blood pressure, heart attack and stroke. Statins stabilize the plaques, making them less prone to rupturing and subsequently forming a dangerous blood clot. Atorvastatin is commercially available for the treatment of primary hypercholesterolemia, dysbetalipoproteinemia and homozygous familial hypercholesterolemia.

Atorvastatin is susceptible to heat, moisture, low pH environment, and light. In an acidic environment, the hydroxy acid moiety present in atorvastatin converts to lactone. In addition, when formulated, atorvastatin may be further destabilized by contact with the molecular moieties of other excipients. Since commonly used excipients, such as binders, diluents, anti-adherents and surfactants may adversely interact with atorvastatin, it is necessary to add a stabilizer to the composition.

Various attempts have been made to stabilize atorvastatin. WO 00/35425 discloses attempts to stabilize statin formulations using buffering agents capable of providing a pH in the range from 7 to 11.

U.S. Pat. Nos. 5,686,104 and 6,126,971 disclose oral pharmaceutical formulations of atorvastatin in which the formulation is stabilized by the addition of a pharmaceutically acceptable alkaline earth metal salt. According to these patents, large amounts of alkaline earth metal salt are required to stabilize the formulation. For example, these patents provide examples in which the drug compositions contain approximately 22% of an alkaline earth metal salt used to stabilize the atorvastatin. Nonetheless, these patents state that between 5% and 75% of the composition can be the alkaline earth metal salt. The alkaline earth metal salt is described as providing effective control of the microenvironment of the composition.

We have now developed an alternate stable oral pharmaceutical composition comprising atorvastatin or a pharmaceutically acceptable salt thereof and an alkalizing agent.

### SUMMARY OF THE INVENTION

In one general aspect, the present invention provides for a stable oral pharmaceutical composition, which includes:
a) atorvastatin or a pharmaceutically acceptable salt thereof; and
b) an alkalizing agent selected from the group consisting of sodium carbonate, sodium bicarbonate, and mixtures thereof;
   wherein the buffering capacity of the composition is between 2 to 5.

Embodiments of this aspect of the invention may include one or more of the following features. For example, the atorvastatin may be present in an amount of from about 2% to about 10% by weight of the composition. The atorvastatin may be present in the formulation as a crystalline Form I atorvastatin.

The pharmaceutical composition may include a mixture of sodium carbonate and sodium bicarbonate. The ratio of sodium carbonate to sodium bicarbonate may be from about 1:3 to about 1:8. The sodium bicarbonate may be present in an amount of from about 2% to about 10% by weight of composition and the sodium carbonate may be present in an amount of from about 0.1% to about 5% by weight of composition.

The one or more antioxidants may be selected from a group consisting of butylated hydroxy anisole, butylated hydroxy toluene, DL-alpha-tocopherol, ascorbic acid, sodium ascorbate, fumaric acid, and mixture thereof.

In another general aspect, the present invention provides for a process of making a stable oral pharmaceutical composition, wherein the process includes the steps of:
a) preparing a dispersion of antioxidant in a suitable solvent;
b) applying the dispersion of step a) to a pharmaceutically acceptable excipient to form a blend;
c) blending atorvastatin with an alkalizing agent and other pharmaceutically acceptable excipients to form a second blend;
d) blending the blend of step b) with the second blend of step c) to form a final blend;
e) lubricating the final blend of step d); and
f) compressing the final blend of step e) into a suitable sized tablet.

### DETAILED DESCRIPTION OF THE INVENTION

Atorvastatin, as used herein, may be present in the form of atorvastatin or pharmaceutically acceptable salts thereof, for example, calcium, magnesium and potassium. Atorvastatin may exist in any of the solid state forms available, such as, amorphous or any other polymorphic form, in particular, crystalline Form I.

Atorvastatin may be present alone or in combination with another active ingredient, for example, amlodipine, ezetimibe, niacin, ramipril, aspirin or other cardiovascular agents.

The term “stable” as used herein, means the pharmaceutical composition of atorvastatin is stable when subjected to the stability conditions of 40° C. and 75% RH for 6 months, wherein the total relative substance is not more than 1%.

Sodium bicarbonate or sodium carbonate as used herein act as alkalizing agents. They improve the stability of a pharmaceutical composition as well as play an important role in achieving the desired in vivo release profile. Sodium bicarbonate may be present in an amount of about 2% to about 10%, in particular, about 3% to about 7% by weight of the composition. Sodium carbonate may be present in an amount of about 0.1% to about 5% by weight of the composition.

The composition may include a mixture of sodium bicarbonate and sodium carbonate as the alkalizing agent. The ratio of sodium carbonate to sodium bicarbonate may vary from about 1:3 to about 1:8.

Buffering capacity denotes the resistance to a change in pH. Buffering capacity of the composition is measured by a method reported by Hawkes et al., in The Chemical Educator, Vol 1, No 8, (1996). To a 1 mg/ml solution of tablet (for 80 mg strength—one tablet of 80 mg strength is dissolved in 80 ml of water), 10 ml of 0.1N HCl is added and the difference in pH is calculated with regard to the initial pH and the pH after the addition of hydrochloric acid. The buffering capacity is calculated by using the following formula:

$$Buffer capacity = \frac{( moles of hydrochloric acid added per liter )}{difference in pH}$$

The pharmaceutical composition may further include one or more additional stabilizer(s). The additional stabilizer may be antioxidants. Examples of antioxidants include butylated hydroxy anisole, butylated hydroxy toluene, DL-alpha-tocopherol, ascorbic acid, sodium ascorbate, fumaric acid, or mixture thereof.

The term “pharmaceutical composition”, as used herein, includes solid dosage forms, such as, tablets, capsules, pills, granules and pellets. Tablets may be present in the form of monolayer tablets or multilayer tablets.

The pharmaceutical composition may further include one or more other pharmaceutically acceptable excipients, for example, disintegrants, binders, diluents, and lubricants/glidants.

Suitable disintegrant(s) include one or more of crospovidone, low-substituted hydroxypropyl cellulose, croscarmellose sodium, sodium starch glycolate, starch and carmellose calcium.

Suitable binders include one or more of methyl cellulose, hydroxypropyl cellulose (HPC-L), methylcellulose, carboxymethyl cellulose sodium, hydroxypropyl methylcellulose, polyvinylpyrrolidone, and mixtures thereof.

Suitable diluents include one or more of lactose, mannitol, microcrystalline cellulose, cellulose powdered, and mixtures thereof.

Suitable lubricants/glidants include one or more of colloidal silicon dioxide, stearic acid, magnesium stearate, calcium stearate, talc, hydrogenated castor oil, sucrose esters of fatty acid, microcrystalline wax, yellow beeswax, white beeswax, sodium stearylfumarate, and mixture thereof.

The tablets may be prepared by a direct compression method or by a granulation process. Granules can be prepared by either dry granulation or wet granulation. Wet granulation may be carried out using a granulating fluid or binder solution. The binder solution may include a suitable hydrophilic polymer dispersed or dissolved in a solvent. Dry granulation may be carried out by roller compaction or slugging.

Solvents used for granulation include one or more of water, ethyl alcohol, isopropyl alcohol, acetone, and mixtures thereof.

The pharmaceutical composition of the invention may optionally be coated with a functional and/or nonfunctional coating. The coating may include one or more coating additives, such as film forming polymers, plasticizers, coloring agents, opacifiers, solvents and lubricants/glidants.

Suitable film-forming polymers include one or more of ethyl cellulose, hydroxypropyl methylcellulose, methylcellulose, carboxy methylcellulose, hydroxymethyl cellulose, hydroxypropyl methylcellulose phthalate, cellulose acetate phthalate, cellulose acetate trimellitate; waxes such as polyethylene glycol; methacrylic acid polymers, such as Eudragit® RL and RS; and gums, such as xanthan gum. Alternatively, commercially available coating compositions comprising film-forming polymers marketed under various trade names, such as Opadry® may also be used for coating.

A polymer solution or dispersion may be prepared in various solvents, including one or more of water, ethanol, isopropyl alcohol, acetone, ether, or mixtures thereof.

Suitable plasticizers include one or more of acetylated triacetin, triethyl citrate, tributyl citrate, glycerol tributyrate, diacetylatedmonoglyceride, polyethylene glycols, propylene glycol, sesame oil, acetyl tributyl citrate, acetyl triethyl citrate, diethyl oxalate, diethyl phthalate, diethyl maleate, diethyl fumarate, dibutyl succinate, diethylmalonate, dioctyl phthalate and dibutyl sebacate. Suitable opacifiers include titanium dioxide.

Suitable coloring agents include one or more of Iron Oxide, Ferric Oxide Yellow, Lake of Tartrazine, Allura Red, Lake of Quinoline Yellow and Lake of Erythrosine.

The composition may be coated using techniques such as spray coating in a conventional coating pan, fluidized bed processor or dip coating.

The pharmaceutical composition may be prepared by a process including the steps of:
a) preparing a dispersion of an antioxidant in a suitable solvent;
b) applying the dispersion of step a) to a pharmaceutically acceptable excipient to form a blend;
c) blending atorvastatin with sodium bicarbonate, sodium carbonate or a mixture thereof and one or more other pharmaceutically acceptable excipients to form a second blend;
d) blending the blend of step b) with the second blend of step c) to make a final blend;
e) lubricating the final blend of step d); and
f) compressing the final blend of step e) into a suitable sized tablet.

The pharmaceutical composition may also be prepared by a process including the steps of:
a) blending atorvastatin with sodium bicarbonate, sodium carbonate or a mixture thereof with one or more pharmaceutically acceptable excipients;
b) optionally, granulating the blend of step a);
c) lubricating the blend of step a) or the granules of step b) to form a second blend; and
d) compressing the second blend of step c) into a suitable sized tablet.

The following examples illustrate the invention but do not limit the scope of the invention.

### Example 1

| Excipients | Qty (mg/tab) |
|---|---|
| Atorvastatin Calcium (Crystalline Form I) | 82.72 |
| Microcrystalline Cellulose (PH 101) | 60.00 |
| Microcrystalline Cellulose (PH 112) | 164.24 |
| Sodium Bicarbonate | 80.00 |
| Lactose Monohydrate | 50.00 |
| Microcrystalline Cellulose (PH 112) | 118.00 |
| Croscarmellose Sodium | 20.00 |
| Colloidal Silicon Dioxide | 14.00 |
| Hydroxypropyl Cellulose-L | 18.00 |
| Croscarmellose Sodium | 84.00 |
| Lactose Monohydrate | 56.76 |
| Microcrystalline Cellulose (PH 112) | 236.88 |
| Lactose Monohydrate | 200.00 |
| BHA | 1.00 |
| BHT | 0.40 |
| IPA | Quantity sufficient |
| Magnesium Stearate | 14.00 |
| Core Total | 1200.00 |
| Opadry ® | 36.00 |
| Water | Quantity sufficient |
| Total | 1236.00 |

### Process:

a) Atorvastatin and microcrystalline cellulose (PH 101) were sifted together.
b) A part of microcrystalline cellulose (PH 112) was sifted separately.
c) Colloidal silicon dioxide, hydroxylpropyl cellulose, croscarmellose sodium, a part of the lactose and another part of the microcrystalline cellulose (PH112) were blended together.
d) BHA and BHT were dissolved in isopropyl alcohol and applied to another part of lactose.
e) Sodium bicarbonate, the remaining part of lactose, the remaining part of microcrystalline cellulose (112) and croscarmellose sodium were sifted together.
f) The blends of step a) and step b) were mixed together.
g) The blend of step e) was mixed with a blend of step f).
h) The blends of step c) and d) were mixed with the blend of step g).
i) The blend of step h) was lubricated with magnesium stearate.
j) The blend of step i) was compressed into a suitable sized tablet.
k) The tablets of step j) were coated with a dispersion of Opadry® in water. The buffering capacity of the composition was measured by the method given in the specification and was found to be 4.79.

### Example 2

| Excipients | Qty (mg/tab) |
|---|---|
| Atorvastatin Calcium | 82.72 |
| Microcrystalline Cellulose (PH 101) | 60.00 |
| Microcrystalline Cellulose (PH 112) | 164.24 |
| Sodium Bicarbonate | 50.00 |
| Sodium Carbonate | 10.00 |
| Lactose Monohydrate | 50.00 |
| Microcrystalline Cellulose (PH 112) | 138.00 |
| Croscarmellose Sodium | 20.00 |
| Colloidal Silicon Dioxide | 14.00 |
| Hydroxypropyl Cellulose-L | 18.00 |
| Croscarmellose Sodium | 84.00 |
| Lactose Monohydrate | 56.76 |
| Microcrystalline Cellulose (PH 112) | 236.88 |
| Lactose Monohydrate | 200.00 |
| BHA | 1.00 |
| BHT7 | 0.40 |
| IPA | Quantity sufficient |
| Magnesium Stearate | 14.00 |
| Core Total | 1200.00 |
| Opadry ® | 36.00 |
| Water | Quantity sufficient |
| Total | 1236.00 |

### Process:

a) Atorvastatin and microcrystalline cellulose (PH 101) were sifted together.
b) A part of the microcrystalline cellulose (PH 112) was sifted separately.
c) Colloidal silicon dioxide, hydroxypropyl cellulose, croscarmellose sodium, a part of the lactose and another part of the microcrystalline cellulose (PH 112) were blended together.
d) BHA and BHT were dissolved in isopropyl alcohol and applied to another part of the lactose.
e) Sodium bicarbonate, sodium carbonate, the remaining part of lactose, the remaining part of microcrystalline cellulose (PH 112) and croscarmellose sodium were sifted together.
f) The blends of step a) and step b) were mixed together.
g) The blend of step e) was mixed with a blend of step f).
h) The blends of step c) and d) were mixed with the blend of step g).
i) The blend of step h) was lubricated with magnesium stearate.
j) The blend of step i) was compressed into a suitable sized tablet.
k) The tablets of step j) were coated with a dispersion of Opadry® in water.

The buffering capacity of the composition was measured by the method given in the specification and was found to be 2.88.

The above composition (Example 2) was packed in desiccant embedded cold form blister pack and in cold form blister pack, which were then subjected to stability studies at 40° C. and 75% RH for six months.

Assay determination was carried out using HPLC method involving C-18 column and mobile phase comprising a mixture of pH 4.0 buffer and organic phase (a mixture of acetonitrile and tetrahydrofuran in the ratio of 925:75).

Relative substance (RS) determination was carried out using a HPLC method involving C-18 column and mobile phase comprising a mixture of Mobile Phases B and C, the composition of which is given below:

Buffer—Ammonium dihydrogen orthophosphate in water.

Mobile Phase A—Mixture of acetonitrile and tetrahydrofuran.

Mobile Phase B—Mixture of buffer and Mobile Phase A.

Mobile Phase C—Mixture of buffer, Mobile Phase A and methanol.

TABLE 1
Stability Data of a Pharmaceutical Composition
of Atorvastatin as Per Example 2

| | | 6 Months | 6 Months |
|---|---|---|---|
| | | (Desiccant-Embedded | (Cold Form |
| | Initial | Cold Form Blister) | Blister) |
| Assay (%) | 99.7 | 99.4 | 99.6 |
| Total RS | 0.146 | 0.656 | 0.709 |

The initial and six months samples were analyzed for assay and total relative substance. The pharmaceutical composition was found to be stable with regard to the relative substances and assay as given in Table 1.

1. A stable oral pharmaceutical composition comprising:
a) atorvastatin or a pharmaceutically acceptable salt thereof; and
b) an alkalizing agent selected from the group consisting of sodium carbonate, sodium bicarbonate, and mixtures thereof;
wherein the buffering capacity of the composition is between 2 to 5.

2. The stable oral pharmaceutical composition according to claim 1, wherein said atorvastatin is present in an amount of from about 2% to about 10% by weight of the composition.

3. The stable oral pharmaceutical composition according to claim 1, wherein said pharmaceutical composition comprises a mixture of sodium carbonate and sodium bicarbonate.

4. The stable oral pharmaceutical composition according to claim 3, wherein the ratio of sodium carbonate to sodium bicarbonate from about 1:3 to about 1:8.

5. The stable oral pharmaceutical composition according to claim 1, wherein said sodium bicarbonate is present in an amount of from about 2% to about 10% by weight of composition.

6. The stable oral pharmaceutical composition according to claim 1, wherein said sodium carbonate is present in an amount of from about 0.1% to about 5% by weight of composition.

7. The stable oral pharmaceutical composition according to claim 1, wherein the pharmaceutical composition may further comprise one or more antioxidants selected from a group consisting of butylated hydroxy anisole, butylated hydroxy toluene, DL-alpha-tocopherol, ascorbic acid, sodium ascorbate, fumaric acid, and mixtures thereof.

8. The stable oral pharmaceutical composition according to claim 1, wherein the atorvastatin is present in the formulation as a crystalline Form I atorvastatin.

9. The stable oral pharmaceutical composition according to claim 1, wherein the pharmaceutical composition is prepared by a process comprising the steps of:
a) preparing a dispersion of antioxidant in a suitable solvent;
b) applying the dispersion of step a) to a pharmaceutically acceptable excipient to form a blend;
c) blending atorvastatin with alkalizing agent and other pharmaceutically acceptable excipients to form a second blend;
d) blending the blend of step b) with the second blend of step c) to form a final blend;
e) lubricating the final blend of step d); and
f) compressing the final blend of step e) into a suitable sized tablet.
